(19) United States
(12) Patent Application Publication
(10) Pub. No.: US 2025/0128072 A1
(43) Pub. Date: Apr. 24, 2025

(54) MANAGING MEDICAL DEVICES USED FOR DETECTING CARDIAC EVENTS

(71) Applicant: Cardiac Pacemakers, Inc., St Paul, MN (US)

(72) Inventors: Lynn Dixon, Lino Lakes, MN (US); Joseph Earl Rogers, JR., Wilmington, NC (US); Julie Lindmark, Shoreview, MN (US)

(21) Appl. No.: 18/635,528

(22) Filed: Apr. 15, 2024

Related U.S. Application Data
(60) Provisional application No. 63/459,758, filed on Apr. 17, 2023.

(51) Int. Cl. A61N 1/365; A61N 1/362; A61N 1/37; A61N 1/372; A61N 1/39

(52) U.S. Cl. CPC A61N 1/365; A61N 1/3621; A61N 1/3702; A61N 1/37247; A61N 1/37264; A61N 1/3925

(57) ABSTRACT

A method includes detecting, using a medical device, a cardiac event based on a comparison of physiological data collected by the medical device to one or more programmed parameters of the medical device. The method further includes detecting, using the medical device, subevents associated with the cardiac event. The method further includes generating, using the medical device, metadata associated with the cardiac event and creating, using a computing system, a timeline of the cardiac event for display. The timeline includes icons associated with the subevents and including at least some of the metadata.

### CROSS REFERENCE TO RELATED APPLICATION

This application claims priority to U.S. patent application 63/459,758, filed on Apr. 17, 2023, which is herein incorporated by reference in the entirety.

### TECHNICAL FIELD

The present disclosure relates generally to approaches for managing medical devices that are used for detecting cardiac events. More specifically, the present disclosure relates to systems, methods, and devices that assist with efficient evaluation and reprogramming of medical devices.

### BACKGROUND

Medical devices can be programmed to sense physiological parameters and provide therapy. However, the initial programming of medical devices may need to be updated if the medical devices are not performing as expected in the field.

### SUMMARY

In Example 1, a method includes detecting, using a medical device, a cardiac event based on a comparison of physiological data collected by the medical device to one or more programmed parameters of the medical device. The method further includes detecting, using the medical device, subevents associated with the cardiac event. The method further includes generating, using the medical device, metadata associated with the cardiac event and creating, using a computing system, a timeline of the cardiac event for display. The timeline includes icons associated with the subevents and including at least some of the metadata.

In Example 2, the method of Example 1, wherein at least one of the subevents includes a sensing event.

In Example 3, the method of any of the preceding Examples, wherein at least one of the subevents includes a therapy event.

In Example 4, the method of any of the preceding Examples, wherein the sensing event includes an onset of the cardiac event.

In Example 5, the method of any of the preceding Examples, wherein the sensing event includes an end of the cardiac event.

In Example 6, the method of any of the preceding Examples, wherein the therapy event includes a shock event.

In Example 7, the method of any of the preceding Examples, wherein the therapy event includes an anti-tachycardia pacing (ATP) event.

In Example 8, the method of any of the preceding Examples, wherein the ATP event includes a burst event or a scan event.

In Example 9, the method of any of the preceding Examples, further including: displaying the timeline on a user display.

In Example 10, the method of any of the preceding Examples, further including: inserting the timeline into a report.

In Example 11, the method of any of the preceding Examples, further including: embedding a hyperlink into the icons.

In Example 12, the method of Example 11, further including: in response to a selection of one of the icons embedded within the hyperlink, displaying a plot of the physiological data associated with the subevent associated with the one of the icons.

In Example 13, the method of Example 12, wherein the plot is included in the report.

In Example 14, the method of any of the preceding Examples, further including: initiating a therapy event based on a comparison of the physiological data to the one or more programmed parameters.

In Example 15, the method of any of the preceding Examples, further including: reprogramming the medical device in response to the timeline, wherein the reprogramming includes altering at least one of the one or more programmed parameters.

In Example 16, a method includes detecting, using a medical device, a cardiac event based on a comparison of physiological data collected by the medical device to one or more programmed parameters of the medical device. The method further includes detecting, using the medical device, subevents associated with the cardiac event. The method further includes generating, using the medical device, metadata associated with the cardiac event and creating, using a computing system, a timeline of the cardiac event for display. The timeline includes icons associated with the subevents and including at least some of the metadata.

In Example 17, the method of Example 16, wherein at least one of the subevents includes a sensing event.

In Example 18, the method of Example 17, wherein the sensing event includes an onset of the cardiac event or an end of the cardiac event.

In Example 19, the method of Example 17, wherein at least one of the subevents includes a therapy event.

In Example 20, the method of Example 19, wherein the therapy event includes a shock event.

In Example 21, the method of Example 19, wherein the therapy event includes an ATP event.

In Example 22, the method of Example 21, wherein the ATP event includes a burst event or a scan event.

In Example 23, the method of Example 16, further including: embedding a hyperlink into the icons.

In Example 24, the method of Example 23, further including: in response to a selection of one of the icons embedded within the hyperlink, displaying a plot of the physiological data associated with the subevent associated with the one of the icons.

In Example 25, the method of Example 16, further including: initiating a therapy event based on a comparison of the physiological data to the one or more programmed parameters.

In Example 26, the method of Example 16, further including: reprogramming the medical device in response to the timeline, wherein the reprogramming includes altering at least one of the one or more programmed parameters.

In Example 27, the method of Example 16, wherein the medical device is a pacemaker or defibrillator.

In Example 28, a system includes a user interface, a first processor, and a first computer-readable medium having a first set of computer-executable instructions embodied thereon. The first set of instructions are configured to be executed by the first processor to cause the first processor to: generate a timeline of a cardiac event for display in the user interface, the timeline including icons associated with subevents of the cardiac event, embed hyperlinks to plots of physiological signals associated with the subevents, and display one of the plots in response to a user selection of one of the icons.

In Example 29, the system of Example 28, wherein at least one of the subevents includes a sensing event and another one of the subevents is a therapy event.

In Example 30, the system of Example 29, wherein the therapy event includes an ATP event.

In Example 31, the system of Example 30, wherein the ATP event includes a burst event or a scan event.

In Example 32, the system of Example 31, wherein the sensing event includes an onset of the cardiac event or an end of the cardiac event.

In Example 33, the system of Example 28, further including: a pacemaker including a second computer-readable medium having a second set of computer-executable instructions embodied thereon. The second set of instructions are configured to be executed by the second processor to cause the second processor to: (1) detect a cardiac event based on a first set of programmed parameters and physiological signals sensed by the pacemaker, (2) detect the subevents associated with the cardiac event, and (3) apply a therapy based on a second set of programmed parameters.

In Example 34, a system includes a medical device and a computing system. The medical device is programmed to: (1) sense physiological signals, (2) detect a cardiac event based on a first set of programmed parameters and the physiological signals, (3) detect subevents associated with the cardiac event, and (4) apply a therapy based on a second set of programmed parameters. The computing system includes a user interface and is programmed to: (1) generate a timeline of the cardiac event for display in the user interface, where the timeline includes icons associated with the subevents, and (2) embed hyperlinks to plots of the physiological signals associated with the subevents.

In Example 35, the system of Example 34, wherein the medical device is a pacemaker or defibrillator.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration of an implantable medical device that delivers electrical simulation to a patient's heart, in accordance with certain instances of the present disclosure.

FIG. 2 is a flowchart illustrating a method of managing medical devices, in accordance with certain instances of the present disclosure.

FIG. 3 is an electrocardiogram plot, in accordance with certain instances of the present disclosure.

FIG. 4 is a timeline of detected cardiac activity, in accordance with certain instances of the present disclosure.

FIG. 5 is a summary page of detected cardiac activity, in accordance with certain instances of the present disclosure.

FIG. 6 schematically shows the summary page of FIG. 5 as well as additional pages with electrocardiogram plot containing detected cardiac events, in accordance with certain instances of the present disclosure.

FIG. 7 shows an electrocardiogram plot with an event indicator, in accordance with certain instances of the present disclosure.

FIG. 8 is a block diagram depicting an illustrative computing device, in accordance with instances of the disclosure.

While the disclosure is amenable to various modifications and alternative forms, specific instances have been shown by way of example in the drawings and are described in detail below. The intention, however, is not to limit the disclosure to the particular instances described. On the contrary, the disclosure is intended to cover all modifications, equivalents, and alternatives falling within the scope of the disclosure as defined by the appended claims.

### DETAILED DESCRIPTION

Medical devices can be programmed to sense physiological parameters and provide therapy. However, the initial programming of medical devices may need to be updated if the medical devices are not performing as expected in the field. For example, medical devices can be programmed to perform functions such as generating an alert, providing responsive therapy, or identifying a potential health event based on one or more programmed settings, such as detection thresholds. But if the programmed thresholds are set such that the medical device is not detecting events or perceiving non-events as events, etc., the medical device may need to be reprogrammed with updated thresholds or other types of parameters. Certain instances of the present disclosure are accordingly directed to systems, methods, and devices that assist with efficient evaluation and reprogramming of medical devices.

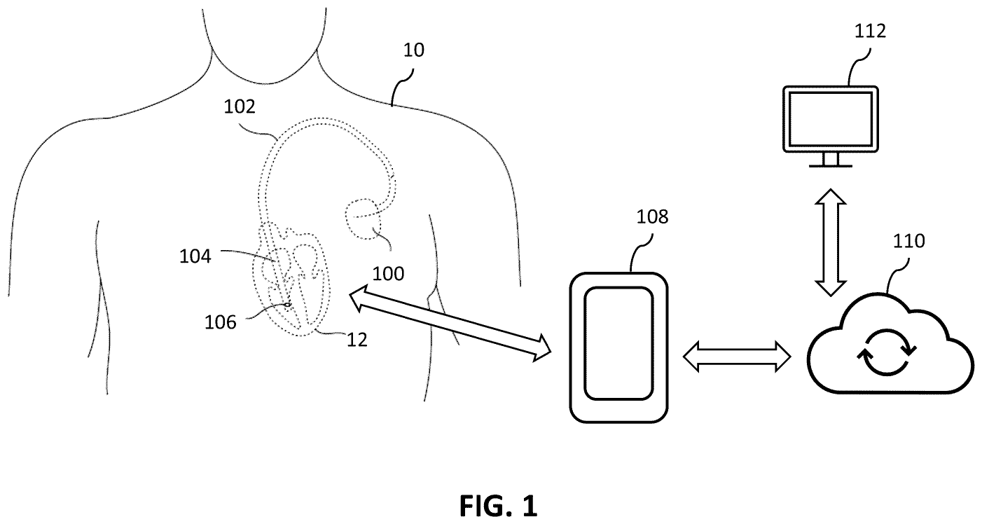

FIG. 1 is a schematic illustration of a medical device 100 that is implanted within a patient 10. The medical device 100 is programmed to sense physiological parameters (e.g., cardiac activity) and/or deliver therapy to the patient's heart 12. For example, the medical device 100 can include one or more leads and/or electrodes that sense electrical activity of the heart 12 and/or provide electrical simulation to the heart 12. Additionally, the medical device 100 can collect and provide a record of the electrical activity of the patient's heart 12.

The medical device 100 of FIG. 1 can be implanted subcutaneously within an implantation location or pocket in the patient's chest or abdomen. The medical device 100 is connected to an implantable lead 102. The lead 102 operates to convey electrical signals between the medical device 100 and the heart 12. The lead 102 can extend into the heart 12 (e.g., through the left brachiocephalic vein and the superior vena cava) such that one or more electrodes 106 disposed on the distal end of the lead 102 can be implanted in the right atrium, right ventricle, left ventricle, or another location. The lead 102 may also be implanted subcutaneously on the patient's chest.

The medical device 100 can sense cardiac activity and generate actionable physiological data. As the physiological data is generated, the physiological data can be monitored by the medical device 100. The medical device 100 can be programmed to detect potential cardiac events (e.g., arrhythmias) based on the physiological data. For example, the medical device 100 can compare the physiological data to various programmed parameters (e.g., thresholds for items such as event duration, heart rate) and, if one or more parameters are met (e.g., thresholds breached), the medical device 100 can determine that a potential cardiac event has occurred.

If the medical device 100 is configured to provide therapy (e.g., an electrical stimulation to the heart 12), the medical device 100 can deliver such therapy in response to the physiological data and the detected potential cardiac event. For example, as will be described in more detail below, the medical device 100 can deliver certain therapy based on how the medical device 100 is programmed to respond to the physiological data and the detected potential cardiac event.

Although the medical device 100 of FIG. 1 is described as a pulse generator (e.g., pacemaker, defibrillator), features described below can be used with other types of implantable medical devices such as cardiac monitors (e.g., implantable and/or wearable diagnostic monitor, implantable loop recorder). Further, although the medical device 100 of FIG. 1 is shown as an implantable medical device, features described below can be used with medical devices that are used when positioned external to the body. For example, medical devices can include electrodes or other features that are coupled to a patient's skin to detect physiological signal such as signals indicative of cardiac activity. As another example, medical devices can include sensors (e.g., acoustic sensors, acceleration sensors) that can detect physiological signal such as signals indicative of cardiac activity, respiration, and patient movement.

A receiver 108 can be communicatively coupled to the medical device 100 and positioned external to the patient's body. The receiver 108 can be a device that is capable of programming, controlling, monitoring, and/or otherwise communicating with the medical device 100. The receiver 108 can help facilitate communication from the medical device 100 to another device or system such as a computing system 110 (e.g., laptop computer, desktop computer, server). The receiver 108 and/or the computing system 110 can be communicatively coupled to a display 112 on which users (e.g., patients, physicians, technicians) can view data generated by the medical device 100.

The medical device 100, the receiver 108, and/or the computing system 100 may be coupled through a communications link such as a short-range radio link (e.g., Bluetooth, IEEE 802.11, or other wireless protocol). The communications link may facilitate bi-directional communication. Data and/or control signals may be transmitted between the medical device 100 and the receiver 108 to coordinate the functions of the medical device 100 and/or the receiver 108. In instances, physiological data (and metadata) may be downloaded from one or more of the medical device 100 and the receiving device 108 periodically or on command. The physician and/or the patient may communicate with the medical device 100 and the receiver 108, for example, to acquire data or to initiate, terminate, or modify (e.g., reprogram) sensing and/or therapy parameters.

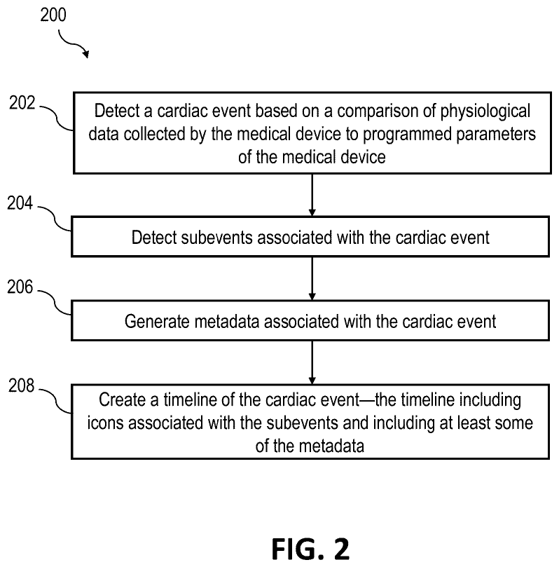

As mentioned above, initial programming of medical devices can be updated if the medical devices are not performing as expected in the field. FIG. 2 outlines a method 200 for assisting with evaluating performance of medical devices and, if desired, reprogramming of medical devices.

The method 200 includes detecting a cardiac event based on a comparison of (1) physiological data collected by a medical device to (2) programmed parameters of the medical device (block 202 in FIG. 2).

The physiological data can be raw data sensed by the medical device such as the medical device of FIG. 1. For example, the physiological data can include or be based on cardiac electrical signals sensed by the medical device.

The programmed parameters can include a wide variety of parameters. For example, a medical device may have 20-30 parameters that can be set and adjusted based on a given patient's history and a physician's recommendations. The parameters can relate to how the medical device monitors the patient's physiological data. For example, the parameters can include sensing-related parameters (e.g., thresholds, values) that are set to—individually or in combination—detect when a patient has potentially experienced a cardiac event. As another example, the parameters can include therapy-related parameters (e.g., thresholds, output values, and action commands) that are set to—individually or in combination—dictate if, when, and how therapy is provided by the medical device after a potential cardiac event has been detected.

When a potential cardiac event is detected (based on one or more programmed parameters), the medical device can begin to store data associated with the detected cardiac event to local memory. In some instances, the medical device is configured to repeatedly delete a certain amount of historical sensed physiological data until a cardiac event is detected. For example, the medical device may continuously delete historical data (that is not associated with a detected event) after expiration of a rolling period of time (e.g., 10-20 seconds) to preserve memory capacity. However, once a potential cardiac event is detected, the medical device can begin storing physiological data until the cardiac event is determined to be complete. This may include storing any physiological data that had not already been deleted as well as a certain amount of physiological data sensed after completion of the detected cardiac event (e.g., 20-60 seconds of physiological data).

As a result, when a potential cardiac event is detected, the medical device may store physiological data associated with the detected cardiac event, including a certain amount of physiological data before and after the onset and completion of the detected cardiac event. In certain instances, completion of the detected cardiac event can be determined once the medical device has not detected an abnormal heartbeat (e.g., as defined by threshold values) after a certain window of time (e.g., a window of 10-60 seconds). This window of time can be different depending on whether therapy was delivered. For example, if a potential cardiac event was detected but did not result in therapy being delivered, the window of time can be less than if the potential cardiac event resulted in therapy. In some instances, a non-therapy event can use a window of 10-20 seconds, and a therapy event can use a window of 20-60 seconds.

Referring back to FIG. 2, the method 200 can further include detecting subevents associated with the cardiac event (block 204 in FIG. 2). For example, an event can include the onset and end of the cardiac event, which are sensing and detection related subevents (e.g., a subevent indicating when the cardiac event began based on programmed parameters).

Additionally, there are therapy related subevents, including but not limited to: ATP (anti-tachycardia pacing), shocks, aborted shocks, ATR (atrial tachycardia response), and SBR (sudden bradycardia response). Such subevents may involve more specific types of therapy such as:
(1) a burst, which is an ATP scheme characterized by one or more attempts of pacing where the pacing cycle length is a percentage or time period (e.g., in order of milliseconds) shorter than a tachycardia cycle length;
(2) a scan, which is another ATP scheme characterized by two or more bursts of pacing where the pacing cycle length of the first burst is a percentage or time period (e.g., in order of milliseconds) shorter than a tachycardia cycle length and the pacing cycle length of subsequent bursts is a percentage or time period (e.g., in order of milliseconds) shorter than the first burst;
(3) a ramp, which is another ATP scheme characterized by one or more bursts of pacing where the pacing cycle length of a first pair of paced beats within the bursts is a percentage or time period (e.g., in order of milliseconds) shorter than a tachycardia cycle length and the pacing cycle length of subsequent bursts is a percentage or time period (e.g., in order of milliseconds) shorter than the first pair of paced beats; and
(4) a ramp-scan, which is another type of ATP scheme characterized by two or more burst pacing combined with attributes of both a scan and ramp.

The method 200 further includes generating metadata associated with the cardiac event (block 206 in FIG. 2). In certain instances, the metadata can relate to how detection of the cardiac event was accomplished and the nature of the therapy (if any) that was delivered by the medical device. Example metadata includes durations associated with the cardiac event/subevents, heart rate, type of cardiac event, therapy information (e.g., charge time, lead impedance, lead polarity, ATP scheme), etc. Such metadata can be automatically generated by the medical device when an event/subevent is detected and also when therapy is provided by the medical device.

As noted above, when the medical device detects a potential cardiac event, the medical device is programmed to store physiological data, detect subevents, and generate and store metadata related to the potential cardiac event (and subevents). The medical device can detect multiple potential cardiac events over time. For implanted medical devices, the medical device can periodically send a data package associated with the detected potential cardiac event to a receiver and/or a computing system positioned external to the patient's body. This data package can be initially stored to local memory in the medical device and can include the underlying physiological data and metadata associated with the detected potential cardiac event.

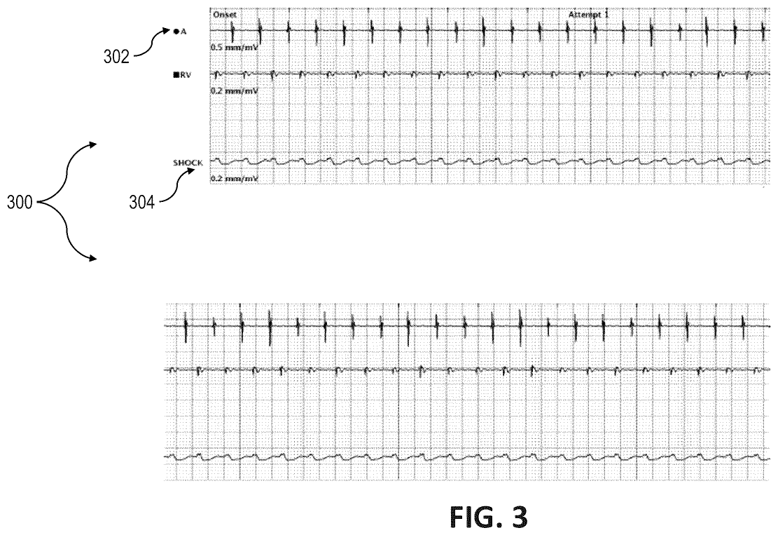

A computing system can receive the data package and process the data for display and review. As one example, FIG. 3 shows a graph 300 with various plots of data. One plot 302 can include the underlying physiological data such as a nearfield electrogram relating to the sensing and detection portion of the medical device. Another plot 304 can include metadata such as a farfield electrogram relating to a therapy portion of the medical device.

However, a single potential cardiac event can be associated with many pages of plots of physiological data and metadata-which, by itself, makes it challenging to evaluate the potential cardiac event efficiently. Further, cardiac events detected by the medical device may ultimately be related to each other, which further adds to the amount of data needing to be reviewed for a given cardiac event. To help address such inefficiencies, the method 200 of FIG. 2 includes automatically creating a timeline of the cardiac event for display (block 208 in FIG. 2).

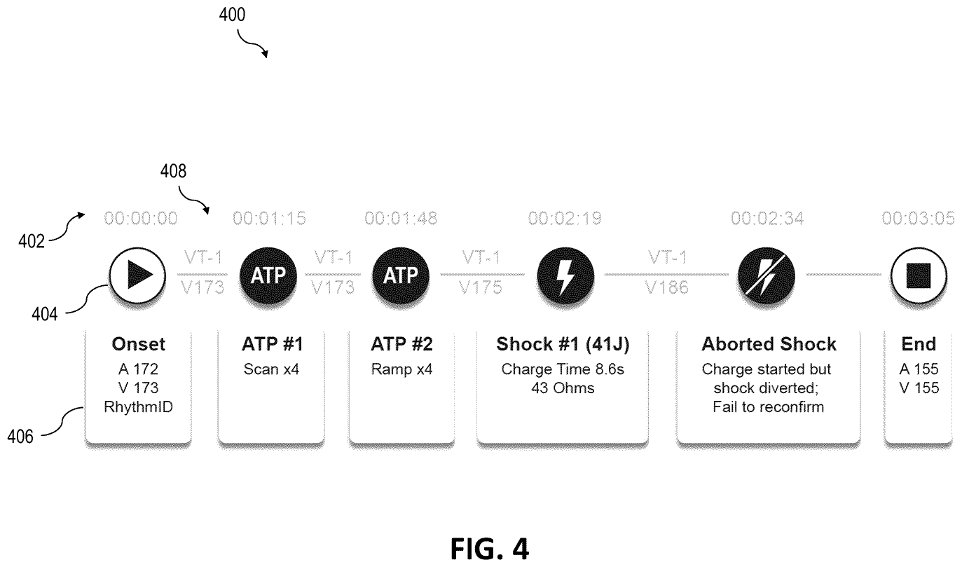

An example event timeline 400 (hereinafter the “timeline 400” for brevity) is shown in FIG. 4. The timeline 400 summarizes key information about a given potential cardiac event such that the potential cardiac event can be efficiently evaluated and, if needed, such that the performance of a medical device can be evaluated and later reprogrammed based on the evaluations. The timeline 400 can be displayed on a display such as the display 112 of FIG. 1.

The timeline 400 can include timestamps 402 that are each associated with a detected subevent. For example, the timestamp 402 associated with the onset subevent can display as time=00:00:00 or a similar indicator.

The timeline 400 can also include icons 404 that are each associated with a detected subevent. The icons 404 can include a visual representation of the detected subevents. For example, the icon 404 associated with the onset subevent can include an arrow that indicates the start of the cardiac event. Other icons 404 can indicate an antitachycardia pacing (ATP) event, a shock event (e.g., a lightning bolt), an aborted shock event (e.g., a lightning bolt with a strikethrough), an end to the cardiac event (e.g., a square), and so on.

In certain instances, a hyperlink can be embedded in one or more of the icons 404 displayed in the timeline 400. For example, when a given hyperlink is selected by a user (via the display 112), the hyperlink can display the underlying data (e.g., an electrocardiogram plot) associated with the selected subevent. This can save users time in combing through pages of an electrocardiogram plot trying to find subevents. In certain instances, displaying the underlying data includes automatically scrolling to the underlying data within a report. In other instances, displaying the underlying data includes generating a pop-up window or display that shows the underlying data.

The timeline 400 can also include short descriptions 406 of each subevent. The descriptions 406 can include metadata associated with subevent as well as relevant decision points (e.g., rhythm ID, fail to reconfirm).

The timeline 400 can also include other metadata 408 displayed between the icons 404. For example, the metadata 408 can include zones and rates (as determined by the medical device) associated with the subevents.

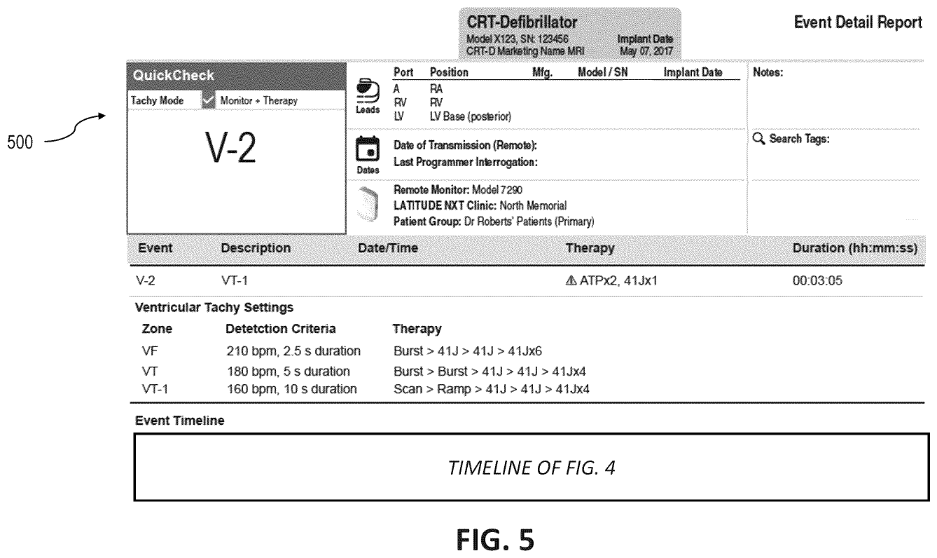

FIG. 5 shows a summary page 500 of detected cardiac activity that incorporates the timeline 400 of FIG. 4. After a computing system generates the timeline 400, the timeline 400 can be inserted into a report (which is discussed in more detail below).

The summary page 500 can include non-physiological patient data (e.g., date of birth, implant date), additional physiological data, additional therapy data, and medical device data (e.g., model, manufacturer) used by the patient. The summary page 500 can be the first page of a report that summarizes and includes a patient's cardiac activity.

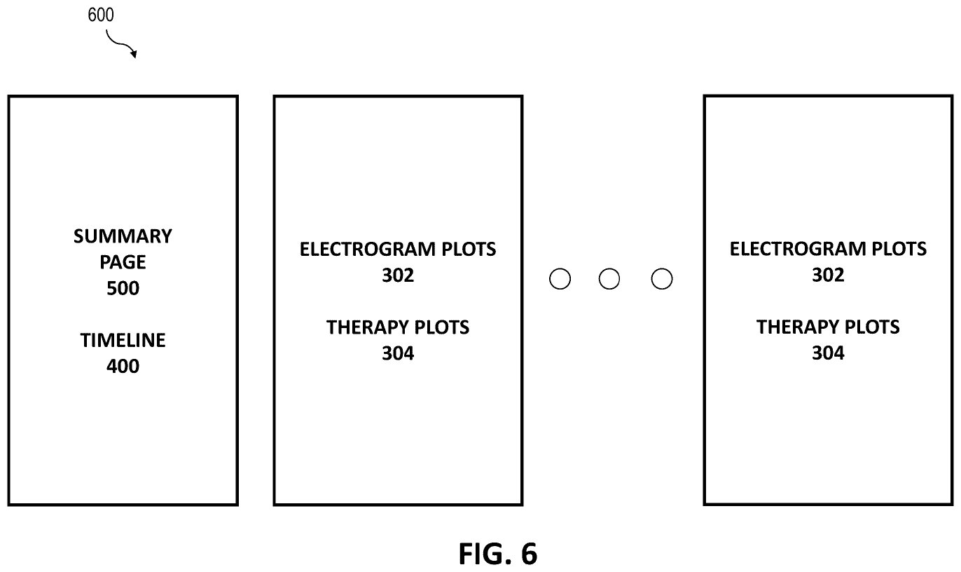

FIG. 6 shows a high-level representation of a report 600, which includes the summary page 500 and timeline 400 which precede pages of plots such as the electrogram plots 302 and therapy plots 304 shown in FIG. 3. The report 600 can be displayed on—and interacted with via—a user display such as a computer monitor, cell phone screen, etc. In certain instances, the report 600 is a portable document format (pdf) that can be opened via a web browser or an application such as Adobe Acrobat or a similar application. Also, in certain instances, the report 600 is included in the patient's electronic medical record.

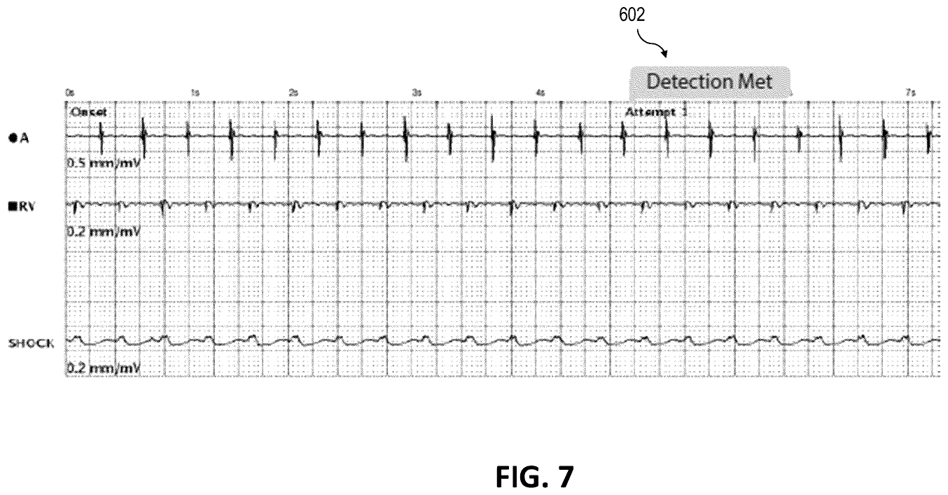

Regardless of the digital format, a user can view and interact with the report 600. For example, the user can select one of the icons in the timeline 400 to utilize an embedded hyperlink to quickly advance (on the display) to the relevant portions of the plots 302/304. The relevant portions of the plots (such as the beginning and end) can be highlighted by one or more event indicators 602 (shown in FIG. 7). The event indicator 602 can be placed and shaped to indicate, for example, when a detected sub-event occurred with respect to the plot(s) in the report 600. The user can then evaluate the underlying data associated with the detected sub-event and determine whether the medical device performed as expected (e.g., whether the cardiac event, sub-event, and/or therapy were appropriately detected and/or administered).

In response to evaluating the detected cardiac event, the user can determine how the medical device should be programmed. For example, based on the information in the timeline 400 and underlying data in the plot(s), the user can determine whether one or more programmed parameters of the medical device should be altered. This may include adjusting a threshold involved in detecting an event/sub-event and/or determining if, when, and how therapy should be delivered.

### Computing Devices and Systems

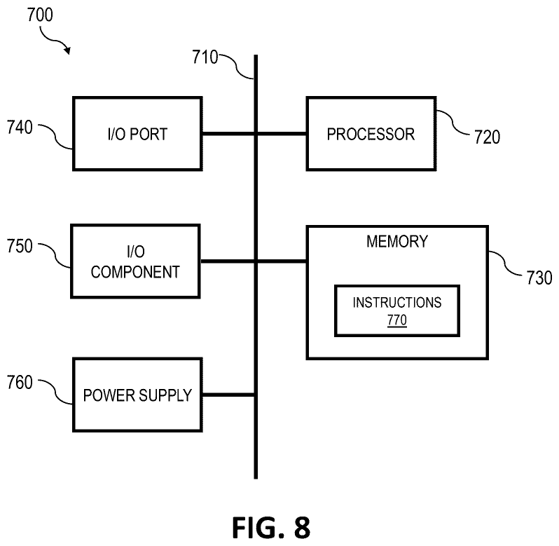

FIG. 8 is a block diagram depicting an illustrative computing device 700, in accordance with instances of the disclosure. The computing device 700 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 8 and described below. For example, the medical device 100, the receiver 108, the computing system 110, and the display 112 (and any computing device connected to the display 112) can each include their own set of components shown in FIG. 8 and described below.

In instances, the computing device 700 includes a bus 710 that, directly and/or indirectly, couples one or more of the following devices: a processor 720, a memory 730, an input/output (I/O) port 740, an I/O component 750, and a power supply 760. Any number of additional components, different components, and/or combinations of components may also be included in the computing device 700.

The bus 710 represents what may be one or more busses (such as, for example, an address bus, data bus, or combination thereof). Similarly, in instances, the computing device 700 may include a number of processors 720, a number of memory components 730, a number of I/O ports 740, a number of I/O components 750, and/or a number of power supplies 760. Additionally, any number of these components, or combinations thereof, may be distributed and/or duplicated across a number of computing devices.

In instances, the memory 730 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 730 stores computer-executable instructions 770 for causing the processor 720 to implement aspects of instances of components discussed herein and/or to perform aspects of instances of methods and procedures discussed herein. The memory 730 can comprise a non-transitory computer readable medium storin the computer-executable instructions 770.

The computer-executable instructions 770 may include, for example, computer code, machine-useable instructions, and the like such as, for example, program components capable of being executed by one or more processors 720 (e.g., microprocessors) associated with the computing device 700. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 770 may be configured to be executed by the processor 720 and, upon execution, to cause the processor 720 to perform certain processes. In certain instances, the processor 720, memory 730, and instructions 770 are part of a controller such as an application specific integrated circuit (ASIC), field-programmable gate array (FPGA), and/or the like. Such devices can be used to carry out the functions and steps described herein.

The I/O component 750 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary instances discussed without departing from the scope of the present disclosure. For example, while the instances described above refer to particular features, the scope of this disclosure also includes instances having different combinations of features and instances that do not include all of the described features. Accordingly, the scope of the present disclosure is intended to embrace all such alternatives, modifications, and variations as fall within the scope of the claims, together with all equivalents thereof.

We claim:

1. A method comprising:
detecting, using a medical device, a cardiac event based on a comparison of physiological data collected by the medical device to one or more programmed parameters of the medical device;
detecting, using the medical device, subevents associated with the cardiac event;
generating, using the medical device, metadata associated with the cardiac event; and
creating, using a computing system, a timeline of the cardiac event for display, the timeline including icons associated with the subevents and including at least some of the metadata.

2. The method of claim 1, wherein at least one of the subevents includes a sensing event.

3. The method of claim 2, wherein the sensing event includes an onset of the cardiac event or an end of the cardiac event.

4. The method of claim 2, wherein at least one of the subevents includes a therapy event.

5. The method of claim 4, wherein the therapy event includes a shock event.

6. The method of claim 4, wherein the therapy event includes an anti-tachycardia pacing (ATP) event.

7. The method of claim 6, wherein the ATP event includes a burst event or a scan event.

8. The method of claim 1, further comprising:
embedding a hyperlink into the icons.

9. The method of claim 8, further comprising:
in response to a selection of one of the icons embedded within the hyperlink, displaying a plot of the physiological data associated with the subevent associated with the one of the icons.

10. The method of claim 1, further comprising:
initiating a therapy event based on a comparison of the physiological data to the one or more programmed parameters.

11. The method of claim 1, further comprising:
reprogramming the medical device in response to the timeline, wherein the reprogramming includes altering at least one of the one or more programmed parameters.

12. The method of claim 1, wherein the medical device is a pacemaker or defibrillator.

13. A system comprising:
a user interface;
a computing device including one or more processors; and
a first computer-readable medium having a first set of computer-executable instructions embodied thereon, the first set of instructions configured to be executed to cause the computing device to:
generate a timeline of a cardiac event for display in the user interface, the timeline including icons associated with subevents of the cardiac event,
embed hyperlinks to plots of physiological signals associated with the subevents, and
display, via the user interface, one of the plots in response to a user selection of one of the icons.

14. The system of claim 13, wherein at least one of the subevents includes a sensing event and another one of the subevents is a therapy event.

15. The system of claim 14, wherein the therapy event includes an anti-tachycardia pacing (ATP) event.

16. The system of claim 15, wherein the ATP event includes a burst event or a scan event.

17. The system of claim 16, wherein the sensing event includes an onset of the cardiac event or an end of the cardiac event.

18. The system of claim 13, further comprising:
a pacemaker including a second computer-readable medium having a second set of computer-executable instructions embodied thereon, the second set of instructions configured to be executed by the second processor to cause the second processor to: (1) detect a cardiac event based on a first set of programmed parameters and physiological signals sensed by the pacemaker, (2) detect the subevents associated with the cardiac event, and (3) apply a therapy based on a second set of programmed parameters.

19. A system comprising:
a medical device programmed to: (1) sense physiological signals, (2) detect a cardiac event based on a first set of programmed parameters and the physiological signals, (3) detect subevents associated with the cardiac event, and (4) apply a therapy based on a second set of programmed parameters; and
a computing system including a user interface and programmed to: (1) generate a timeline of the cardiac event for display in the user interface, the timeline including icons associated with the subevents, and (2) embed hyperlinks to plots of the physiological signals associated with the subevents.

20. The system of claim 19, wherein the medical device is a pacemaker or defibrillator.
